(19) United States
(12) Patent Application Publication
(10) Pub. No.: US 2010/0222880 A1
(43) Pub. Date: Sep. 2, 2010

(54) Breast Prosthesis

(75) Inventors: Dirk Muscat, Ottobrunn (DE); Stefan Moser, Nussdorf am Inn (DE)

Correspondence Address: DILWORTH & BARRESE, LLP, 1000 WOODBURY ROAD, SUITE 405, WOODBURY, NY, 11797, US

(73) Assignee: AMOENA MEDIZIN-ORTHOPAEDIE-TECHNIK GMBH, Raubling (DE)

(21) Appl. No.: 12/537,615

(22) Filed: Aug. 7, 2009

(30) Foreign Application Priority Data
Dec. 2, 2008 (DE) 102008060087.3

(51) Int. Cl. A61F 2/52; D06P 7/00

(52) U.S. Cl. 623/7; 8/497

(57) ABSTRACT

This invention relates to a breast prosthesis which consists of a cup-shaped body of a non-rigid plastic material, which is welded into plastic films to obtain one or more chambers. In accordance with the invention, the filling material is colored with polymeric dyes. This invention furthermore relates to a method for coloring breast prostheses.

### BACKGROUND OF THE INVENTION

This invention relates to a breast prosthesis and a method for manufacturing such breast prosthesis.

Extracorporeal breast prostheses, which are used after a mastectomy, serve different purposes, such as weight compensation. In the manufacture of modem prostheses, the appearance of the finished prosthesis is a very important aspect. Beside the practical properties, which are determined by the main components, especially the coloring of the prosthesis is an essential criterion. Coloring substantially is understood to be the color tone as such.

The color of current breast prostheses primarily depends on the basic skin colors of the wearers. For Europe, a soft pink is chosen. For the Afro-American skin type, for instance, brown and black is chosen. Coloring breast prostheses is effected by admixing corresponding pigments. This is described for instance in U.S. Pat. No. 5,738,812. WO 00/53129 likewise describes the coloring of breast prostheses with coloring agents, wherein here examples for the use of color pigments are provided. UK 1,582,081 likewise discloses the coloring of breast prostheses with pigments. Here, coloring with dyes also is indicated as an alternative, but the same is not explained in greater detail.

Beside the direct coloring, another requirement for coloring breast prostheses is the color uniformity achieved. Coloring prostheses with pigments repeatedly has led to quality problems. When breast prostheses, in particular silicone-filled breast prostheses, are colored by means of pigments, there is regularly obtained a non-uniform color image. Very different phenomena are responsible for this. The non-uniformity of the color is at least partly due to demixing conditions. In addition, the static charge of the pigments can lead to the fact that during filling the same are oriented in a preferred filling path and therefore lead to the non-uniform coloring of the breast prosthesis. Another undesired phenomenon during coloring consists in that the fillers partly settle down.

The use of organic dye molecules, as it is known from the prior art for coloring breast prostheses, leads to basically uniform coloring. In the polymer gel, in particular in the silicone gel, the organic dye molecules tend to migrate, however, and can diffuse through the porous film which surrounds the breast prosthesis and thus discolor the skin or clothing of the wearer. Examples for dyes migrating in the silicone gel body are direct dyes, dispersed dyes or also reactive dyes.

### SUMMARY OF THE INVENTION

Proceeding from a generic breast prosthesis, it therefore is the object of the invention to achieve a uniform coloring of the breast prosthesis, wherein even after corresponding coloring, a change in color while wearing the breast prosthesis is avoided.

In accordance with the invention, this object is solved by the combination of features herein. Here, a breast prosthesis is provided, which is created from a cup-shaped body of a non-rigid plastic material, which is welded into plastic films to obtain one or more chambers, in which the filling material is colored with polymeric dyes. The inventors surprisingly have found that organic dyes, which are functionalized with a polymer, are bound properly in the plastic matrix, i.e. for instance the silicone gel matrix. The dye molecules thus are prevented from migrating through the film. Due to the polymeric residue, the organic dye quite obviously is bound completely in the gel matrix by van der Waals forces, and due to their size the polymeric dye molecules only can diffuse very slowly, if at all, through the silicone gel, but not through the porous outer film.

Polymeric dyes have the advantage that they are miscible and that therefore the color of the breast prostheses can individually be adapted to the color tone of the skin of the wearer. For this purpose, the primary colors red, yellow and blue are mixed in a simple dosing method and filled into the breast bag as colored silicone by means of a suitable dosing and mixing technique.

Preferred aspects of the invention can be taken from the description herein.

Accordingly, the polymeric dyes can be water-soluble or oil-soluble. At least one of the polymeric dyes can be functionalized by means of a polyalkylene oxide chain. Particularly advantageously, the polymeric dye is functionalized by means of a polysiloxane-polyalkylene oxide chain.

In a particularly advantageous way, a particularly uniform color is achieved when hollow microbeads or microspheres made of plastics are used at the same time. Examples for such polymeric dyes are disclosed already in EP 0864617 A1.

The aforementioned invention is also solved by a manufacturing method according to the description herein. For manufacturing breast prostheses from a cup-shaped body of an addition-crosslinking two-component silicone rubber composition, which is welded into plastic films to obtain one or more chambers, the polymeric dye is mixed with the addition-crosslinking two-component silicone rubber composition to adjust the desired color tone and subsequently filled into a film bag clamped into a mold and degassed and cured in the mold.

This method is configured particularly advantageously In that a filler of low density, which consists of hollow spheres and/or microspheres, is admixed to the addition-crosslinking two-component silicone rubber composition.

In this method, one of the polymeric dyes advantageously can be functionalized by means of a polyalkylene oxide chain. Advantageously, this again is a polysiloxane-polyalkylene oxide chain.

### DESCRIPTION OF THE PREFERRED EMBODIMENTS

Further features, details and advantages of the invention can be taken from the examples represented below. In one embodiment, polymeric dyes of the firm Milliken are used. The dye of the invention consists of a mixture of polymeric dyes of the type Liquitint, and here preferably mixtures similar to the skin tone are chosen.

In the correspondingly colored breast prostheses, there is no diffusion of the dyes through the porous film.

The total amount of dye in the basic formulation generally is less than 1.5%.

The silicone test preparations used are based on the following basic formulation:
50 parts: consisting of Dow Coming silicone formulation 7-2962:
   60 parts of component A
   100 parts of component B
50 parts consisting of:
   100 parts: silicone oil
   3 parts: hollow plastic microspheres

In the final analysis, uniformly colored and color-fast breast prostheses can be manufactured with this embodiment.

In a comparative example, organic dye molecules were used for coloring a breast prosthesis with the same starting material. For this purpose, the dye of the type ColyPlast of the firm DyStar was used. Here as well, the amount to be added to the basic formulation was chosen <1%. In this comparative example, there was a diffusion of the dye molecules through the porous film of the breast prosthesis, so that the skin or clothing of the wearer was discolored.

1. A breast prosthesis, consisting of a cup-shaped body of a non-rigid plastic material, preferably an addition-crosslinking two-component silicone rubber composition, which is welded into plastic films to obtain one or more chambers, wherein the filling material is colored with polymeric dyes.

2. The breast prosthesis according to claim 1, wherein the polymeric dyes are water-soluble.

3. The breast prosthesis according to claim 1, wherein polymeric dyes are oil-soluble.

4. The breast prosthesis according to claim 1, wherein at least one of the polymeric dyes is functionalized by a polyalkylene oxide chain.

5. The breast prosthesis according to claim 1, wherein at least one of the polymeric dyes is functionalized by a polysiloxane-polyalkylene oxide chain.

6. A method for manufacturing breast prostheses from a cup-shaped body of an addition-crosslinking two-component silicone rubber composition, which is welded into plastic films to obtain one or more chambers, wherein that polymeric dyes are mixed with the addition-crosslinking two-component silicone rubber composition to adjust the desired color tone and subsequently are filled into a film bag clamped into a mold and are degassed and cured in the mold.

7. The method according to claim 6, wherein a filler of low density, which consists of hollow spheres and/or microspheres, is admixed to the addition-crosslinking two-component silicone rubber composition.

8. The method according to claim 6, wherein at least one of the polymeric dyes is functionalized by a polyalkylene oxide chain.

9. The method according to claim 6, wherein at least one of the polymeric dyes is functionalized by a polysiloxane-polyalkylene oxide chain.

10. The breast prosthesis according to claim 2, wherein at least one of the polymeric dyes is functionalized by a polyalkylene oxide chain.

11. The breast prosthesis according to claim 3, wherein at least one of the polymeric dyes is functionalized by a polyalkylene oxide chain.

12. The breast prosthesis according to claim 2, wherein at least one of the polymeric dyes is functionalized by a polysiloxane-polyalkylene oxide chain.

13. The breast prosthesis according to claim 3, wherein at least one of the polymeric dyes is functionalized by a polysiloxane-polyalkylene oxide chain.

14. The method according to claim 7, wherein at least one of the polymeric dyes is functionalized by a polyalkylene oxide chain.

15. The method according to claim 6, wherein at least one of the polymeric dyes is functionalized by a polysiloxane-polyalkylene oxide chain.
